(19) United States
(12) Patent Application Publication
(10) Pub. No.: US 2009/0143619 A1
(43) Pub. Date: Jun. 4, 2009

(54) PROCESS FOR THE PRODUCTION OF PHOSGENE WITH REDUCED CO EMISSION

(75) Inventors: Hermann Kauth, Krefeld (DE); Ulrich Blaschke, Krefeld (DE); Wilfried Kaschube, Monheim (DE); Klaus Kebler, Toenisvorst (DE); Christian Kords, Krefeld (DE); Thomas Elsner, Duesseldorf (DE)

Correspondence Address: CONNOLLY BOVE LODGE & HUTZ, LLP, P O BOX 2207, WILMINGTON, DE, 19899, US

(73) Assignee: Bayer MaterialScience AG, Leverkusen (DE)

(21) Appl. No.: 12/275,810

(22) Filed: Nov. 21, 2008

(30) Foreign Application Priority Data
Nov. 29, 2007 (DE) 102007057462.4

(51) Int. Cl. C07C 51/58

(52) U.S. Cl. 562/847

(57) ABSTRACT

The application relates to a process for the continuous production of phosgene from chlorine and CO with reduction of the carbon monoxide emission (CO emission).

### RELATED APPLICATIONS

This application claims benefit to German Patent Application No. 10 2007 057 462.4, filed Nov. 29, 2007, which is incorporated herein by reference in its entirety for all useful purposes.

### BACKGROUND OF THE INVENTION

The application relates to a process for the continuous production of phosgene from chlorine and CO with reduction of the carbon monoxide emission (CO emission).

CO emissions are increasingly gaining importance in the course of capacity expansions in phosgene-processing production plants.

The continuous production of phosgene in so-called phosgene generators—hereafter also abbreviated to generators—is known and described in detail in its basic principles, for example in “Ullmann”, 5^{th} edition, volume 19, pp 411 ff, section 3. “Production”.

A gaseous mixture of CO and chlorine is therein continuously passed over granulated activated carbon in a phosgene generator in an approximately stoichiometric ratio, wherein the CO is added in slight excess to avoid larger residual chlorine contents in the phosgene formed. A particularly pure phosgene which is required to produce high-quality plastics, such as for example polycarbonates or polyurethanes from diisocyanates, is obtained by selective condensation of the crude phosgene obtained from the phosgene generator. Low-boiling by-products, such as for example CO and other residual gases, are therein separated off and discharged from the system via the exhaust air route. In larger, multiply available and optionally interconnected plants of this type, quite noteworthy levels of CO emissions or CO gas concentrations which can become a problem with regard to environmental impact and also with regard to the formation of explosive gas mixtures with atmospheric oxygen and must therefore be avoided, are produced with this procedure in continuous operation.

The object on which the present invention was based accordingly consisted in clearly reducing CO emissions in the production of phosgene from chlorine and CO by suitable, preferably technically simple and cost-effective measures.

### EMBODIMENTS OF THE INVENTION

An embodiment of the present invention is a process for continuously producing phosgene, comprising producing phosgene from CO and chlorine in the presence of at least one catalyst in at least one generator, condensing said phosgene in a condenser, separating off CO-containing residual gas, reacting said CO-containing residual gas with chlorine in a secondary generator to produce phosgene, condensing said phosgene produced in said secondary generator in a secondary condenser, and separating off uncondensed residual gas.

Another embodiment of the present invention is the above process, wherein addition of chlorine into said secondary generator is controlled by analysis of the CO content of the CO-containing residual gas.

Another embodiment of the present invention is the above process, wherein addition of chlorine into said secondary generator is controlled such that the gas mixture added to said secondary generator has a CO excess of at least 1.5 volume % based on added chlorine.

Another embodiment of the present invention is the above process, wherein said uncondensed residual gas is discharged from the process and passed to a phosgene decomposition unit.

Another embodiment of the present invention is the above process, wherein said catalyst is activated carbon and/or coke.

Another embodiment of the present invention is the above process, wherein said generators and/or secondary generators are heat exchangers in the form of tubular reactors.

Another embodiment of the present invention is the above process, wherein said tubular reactors have parallel vertically arranged reaction tubes wherein a coolant circulates in the coolant chamber laterally surrounding the reaction tubes and which terminate at their upper and lower end in each case in an upper and lower gas chamber of the tubular reactor, wherein the reaction tubes of the tubular reactor are filled with activated carbon and the upper gas chamber of the tubular reactor additionally contains on the reaction tubes a layer of activated carbon and/or coke.

Another embodiment of the present invention is the above process, wherein said reaction tubes are filled at the lower end with catalytically non-active materials and on them have a layer of activated carbon.

Another embodiment of the present invention is the above process, wherein several generators are used in parallel operation.

Another embodiment of the present invention is the above process, wherein said phosgene condensed in said condenser and in said secondary condenser is collected in a common liquid phosgene receiver.

### BRIEF DESCRIPTION OF THE DRAWING

FIG. 1 illustrates a particularly preferred embodiment of the process according to the invention.

### DESCRIPTION OF THE INVENTION

Surprisingly, it was found that the reaction of the CO-containing residual gases from the condenser(s) of the crude phosgene obtained in the reaction with chlorine and CO to phosgene clearly reduces the CO emission with addition of further chlorine in at least one secondary generator to further phosgene.

The present invention accordingly provides a process for the continuous production of phosgene, wherein phosgene is produced from CO and chlorine in the presence of at least one catalyst in at least a first generator, then the phosgene is condensed in at least a first condenser and CO-containing residual gases are separated off, characterised in that the CO-containing residual gases from the first condenser(s) are reacted to further phosgene with addition of further chlorine in at least one secondary generator and then the phosgene produced in the secondary generator is condensed in at least one secondary condenser and uncondensed residual gases are separated off.

The addition of further chlorine for the reaction in the secondary generator(s) is preferably controlled by analysis of the CO content of the CO-containing residual gases separated off in the first condenser(s). The addition of further chlorine for the reaction in the secondary generator(s) is particularly preferably controlled such that in the gas mixture which is added to the secondary generator(s) there is a CO excess of at least 1.5 vol. % based on added chlorine.

The uncondensed residual gases separated off in the secondary condenser(s) are preferably discharged from the process and passed to the phosgene decomposition unit.

Activated carbon and/or coke is preferably used as catalyst. Activated carbon with a specific surface area of at least 500 m²/g, preferably more than 900 m²/g, and a pore volume for pore diameters of 1 to 100 nm of at least 0.06 ml/g, preferably more than 0.07 ml/g, is thereby particularly preferred. The activated carbon is preferably used in granulate form. Granulates with a diameter of between 3 and 8 mm, especially preferably between 3 and 5 mm, are particularly preferred. By-product broken coke can, for example, be used as coke.

Preferably heat exchangers, particularly preferably heat exchangers in the form of tubular reactors, are used as generators and/or secondary generators. One or more such generators and/or secondary generators can be used. These can be operated arranged in series or parallel. In preferred embodiments, several generators and/or several secondary generators, in particular several generators in parallel operation can be used. For industrial application, the reaction of chlorine and CO is preferably carried out in one or more generators in the form of towers—so-called phosgene generator towers. In preferred embodiments, several such towers in parallel operation can be used. Heat exchangers, preferably in the form of towers, are preferably tubular reactors which contain a multiplicity of reaction tubes with a diameter of maximum 70 mm, preferably of maximum 60 mm, particularly preferably of maximum 50 mm. These reaction tubes are filled with the catalyst for the reaction of CO and chlorine to phosgene. The reaction tubes are preferably arranged vertically through which a coolant circulates in the coolant chamber laterally surrounding the reaction tubes and the reaction tubes terminate at the top and bottom in each case in a gas chamber of the tubular reactor which is separated from the coolant chamber and in which the gases flow together or are distributed on to the individual tubes. Such tubular reactors are known to the person skilled in the art. Filling of the reaction tubes with the catalyst is carried out preferably and advantageously such that the pressure loss through each individual reaction tube of the tubular reactor is virtually identical. This allows an even throughflow of all reaction tubes during operation of the generator.

In the process according to the invention, the gases CO and chlorine are mixed before entry into the lower gas chamber of the tubular reactor, preferably by static mixers, and the gas mixture passed from bottom to top through the reaction tubes. The phosgene formed is taken off at the head of the upper gas chamber of the tubular reactor.

A particularly suitable material for such tubular reactors is normal structural steel.

The temperatures inside the catalyst contact, preferably activated carbon contact, produced in the reaction of CO with chlorine can be up to 500° C. The high quantities of heat liberated must therefore be very effectively led off in order to safely avoid partial overheatings of the reactor. This applies in particular for the lower area of the reactor where there is chlorine that has not yet reacted. Overheatings could produce so-called chlorine-iron fire there. It can therefore be of advantage to fill the reaction tubes at the lower end of the tubular reactor, i.e. where the CO-chlorine mixture enters, preferably with catalytically non-active materials, such as e.g. with ceramic saddle packings, and only thereby fill the reaction tubes with the catalyst. This filling with catalytically non-active materials can be carried out up to a height of a few centimetres.

The reaction tubes are arranged in the tubular reactor such that a liquid cooling medium adequately flows around them so that the heat of reaction liberated can be led off sufficiently quickly on the formation of phosgene from chlorine with CO. There are various methods for leading off or using the heat of reaction in the combination of chlorine with CO.

In processes with cooling temperatures above 100° C., part of the heat of reaction is used at a high temperature level with indirect heat exchangers to produce vapour. The hot reaction gases from the first generator are reacted in a second generator at lower temperatures such that the free chlorine content in the phosgene is as low as possible. Such a process is known to the person skilled in the art and is described in detail for example in DE-A 33 27 274.

Other processes work at cooling temperatures below 100° C. and use either a vapour cooling by evaporating a heat carrier in an indirectly cooled vapour cooling system or a conventional liquid cooling which likewise uses indirect cooling systems. Such processes are known to the person skilled in the art.

For the process according to the invention, both cooling processes at cooling temperatures above 100° C. and those at cooling temperatures below 100° C. can be used. A cooling process at cooling temperatures below 100° C. is preferably used, particularly preferably one that uses a conventional liquid cooling. Water, in particular deionised water (DI water), or dilute aqueous alkali solution, in particular an alkali solution with a pH of 8 to 10, thereby preferably serves as cooling medium. The dilute aqueous alkali solution can for example be a dilute aqueous NaOH or KOH solution. The aqueous cooling medium is for safety reasons preferably part of a closed system with a balancing tank and circulating pump, the closed-circuit condenser of which is indirectly cooled from outside.

In cooling with aqueous cooling mediums, the gas pressure in the reaction tubes of the tubular reactor is particularly preferably always higher than the pressure in the aqueous coolant system in order to avoid the penetration of water into the reaction tubes in the event of leakage, because such penetration could lead to dangerous secondary reactions.

In the coolant system, the pH and/or the electrical conductivity can furthermore preferably be constantly monitored by means of a pH electrode or conductivity probe in order to detect the penetration of phosgene into the coolant system and thus detect leaks at an early stage.

The coolant system is preferably connected via the balancing tank at controlled nitrogen overpressure to the phosgene decomposition unit. The coolant system flows though the area around the reaction tubes preferably from bottom to top. The coolant flow is thereby particularly preferably linked by suitably arranged partitions between the tubes such that there is a constant change between tangential and vertical flow on the reaction tubes.

The process according to the invention is preferably operated with raw materials of high purity because the purity of the raw materials has an effect on the procedure of the process and the quality of the phosgene formed. A high purity of CO and chlorine gas makes it possible to meet the high purity requirements of the phosgene formed therefrom which are prescribed by the required quality demands of the polymers to be produced therefrom.

CO gas preferably used is desulfurised and contains no more than 5 mg/m³ sulfur, preferably no more than 2 mg/m³ sulfur—sulfur content based on the volume of the CO gas—of organic or inorganic sulfur compounds. The formation of sulfur chlorides as by-products of the phosgene in the generator which in the subsequent polymer syntheses can enter into undesired secondary reactions, can therewith be avoided or clearly reduced. Since methane forms undesirable carbon tetrachloride with chlorine at the temperatures prevalent in the generator, the methane content in the CO gas used is preferably not greater than 50 vol. ppm, particularly preferably not greater than 30 vol. ppm. The hydrogen content in the CO gas used is preferably less than 2.0 vol. %, preferably less than 1.5 vol. %, because larger quantities of hydrogen in the CO gas used can possible react with chlorine uncontrollably to hydrogen chloride (chlorine detonating gas reaction) which can in addition also have corrosive effects on the apparatus materials. The oxygen content in the CO gas used is preferably less than 0.15 vol. % to avoid explosive gas mixtures. The water content in the CO gas is preferably less than 10 mg/m³, as a result of which secondary reactions and corrosions in the plant parts can be avoided. The CO gas used can be obtained for example by partial oxidation of carbon supports, such as for example described in DE-A 103 48 116, and subsequent desulfurisation, as described for example in EP-A 1 590 295, or it can be removed from the reformer process for methane after the gas purification steps. The reformer process is known to the person skilled in the art.

The chlorine gas used contains, for the same reasons as stated above for CO gas, preferably less than 0.1 vol. % oxygen and preferably less than 250 mg/m³ water. The chlorine gas can be obtained for example by sodium chloride electrolysis or by hydrogen chloride electrolysis. Preferred electrolysis processes are the membrane process and the amalgam process. These electrolysis processes are known to the person skilled in the art. The chlorine gas contains preferably less than 80 mg/m³ bromine and preferably less than 10 mg/m³ nitrogen trichloride.

In order to achieve as complete a reaction of chlorine as possible or as low a residual chlorine content as possible in the phosgene to be produced at the optimum reaction temperatures, the reaction of CO and chlorine is preferably carried out with an excess of CO based on chlorine of at least 1.1 vol. %. The outlet temperature of the phosgene gas at the head of the generator must be kept as low as possible because as the temperature rises, the reaction equilibrium is further displaced towards the educts. Thus for example at 200° C., approx. 0.4 vol. % of the phosgene is dissociated in CO and chlorine, at 100° C. approx. 50 ppm of the phosgene. It can therefore be of advantage to construct and operate the generator such that the phosgene outlet temperature is preferably below 75° C., particularly preferably below 65° C., in order to preferably achieve residual chlorine contents in the phosgene to be produced of less than 5 vol. ppm, particularly preferably of less than 1 vol. ppm, based on the total volume of the phosgene. This can be achieved for example by limiting the throughput of the gases through the reactor or by an adequate dimensioning of the reaction tubes. The crude phosgene produced in this way can also contain, in addition to the excess quantities of CO produced, inert gases such as e.g. nitrogen and/or carbon dioxide, and optionally traces of chlorine, hydrogen chloride, hydrogen, carbon tetrachloride etc.

The condensable gaseous minor components of the crude phosgene are separated off in the process step in the first phosgene condenser following the reaction of CO and chlorine in the generator. For this, the crude phosgene is preferably condensed at temperatures of below −10° C. and atmospheric pressure or slight overpressure up to 3 bar (absolute), collected in a cooled liquid phosgene receiver and from there passed for further reaction to appropriate applications. A heat exchanger, in preferred embodiments one made of high-grade steel, with refrigerating brine, for example, is suitable as condenser. Such condensers are known to the person skilled in the art. The original crude phosgene is purified by this separation of the volatile components which do not condense at these temperatures. It can be used at this purity to produce polymers or the precursors thereof.

The first phosgene condenser is connected on the off gas side to at least one (phosgene) secondary generator into which the gases not condensable at these temperatures are led off. These volatile components contain, at a significant level, the CO excess not reacted in the production of phosgene in the first generator. This residual gas stream from the first condenser containing the unreacted CO excess is mixed in a subsequent process step in at least one secondary generator with the required quantities of chlorine and reacted to further phosgene. The addition of the required quantities of chlorine for the reaction in the secondary generator(s) is thereby preferably controlled by analysis of the CO content of the CO-containing residual gases separated off in the first condenser(s).

The secondary generator(s) are in principle of the same construction as the first generator(s) which have already been described for the first step of phosgene production. Here again, the use of a secondary generator or several secondary generators, preferably in parallel operation, is possible. Dimensioning of the secondary generator(s) is such that even extremely different flow rates of off gas from the first condenser(s) can be reacted without problem. The secondary generator(s) can be cooled in the same way as for the first generator(s). The secondary generator(s) are, because of the relatively small phosgene conversion, preferably operated at cooling temperatures of less than 100° C. by conventional liquid cooling with aqueous media as coolant, wherein here again there is preferably an indirect cooling system.

The CO content of the off gas from the first condenser is monitored continuously. The added quantity of chlorine gas in the secondary generator(s) is controlled accordingly such that preferably a CO excess based on chlorine of at least 1.5 vol. %, preferably of at least 2 vol. %, is present in the gas mixture passed to the secondary generator. Also in the phosgene which is led off from the secondary generator, it is desirable to keep the residual chlorine content as low as possible for the reasons already mentioned. The secondary generator is therefore also preferably operated such that the phosgene outlet temperature at the head of the secondary generator is preferably below 75° C., particularly below 65° C.

The phosgene produced in the secondary generator(s) is condensed in at least one (phosgene) secondary condenser, preferably at temperatures below −10° C., at atmospheric pressure or slight overpressure up to 3 bar (absolute), and thus separated from the residual gases still remaining. The heat exchangers already described as first condensers, for example, are suitable as secondary condensers. The residual gases separated off are then passed preferably directly to the phosgene decomposition unit in order to be completely and safely destroyed there. The level of unreacted CO gas contained in the residual gases from the secondary condenser(s) is substantially lower compared to the CO level in the residual gas from the first condenser(s).

The phosgene liquefied in the secondary condenser is passed to a liquid phosgene receiver. The phosgene condensed in the first condenser(s) and the secondary condenser(s) is preferably passed to a common liquid phosgene receiver. The CO excess is therewith for the most part converted to acceptable phosgene.

The phosgene decomposition unit works according to known process principles described for example in “Ullmann”, 5^{th} edition, volume 19, pp 411 ff, section 6 “Waste-Gas Treatment”. In a preferred process variant, phosgene decomposition is carried out on activated carbon with water.

The process according to the invention offers a technically simple and cost-effective option to reduce CO emission because on the one hand no new process engineering with foreign product streams is required and optionally existing apparatus or production plants can be used, and because on the other hand the excess CO is reacted to usable phosgene.

Surprisingly, it was furthermore found that the reaction of CO with chlorine in the process according to the invention can be clearly improved and in this way furthermore the required CO excess quantities can be clearly reduced if filling the tubular reactor with the catalyst, preferably activated carbon, is done in such a way that not only are the reaction tubes evenly filled with activated carbon, but also in the upper chamber above the reaction tubes in which the reaction gases are again reunited, there is a layer of activated carbon and optionally coke.

In particularly preferred embodiments of the process according to the invention, therefore, the tubular reactors used have parallel, vertically arranged reaction tubes in which a coolant circulates in the coolant chamber laterally surrounding the reaction tubes and which terminate at their upper and lower end in each case in an upper and lower gas chamber of the tubular reactor, wherein the reaction tubes of the tubular reactor are filled with activated carbon and the upper gas chamber of the tubular reactor additionally contains over the reaction tubes a layer of activated carbon and/or coke. This layer of activated carbon and optionally coke preferably has a thickness of at least 10 cm, particularly preferably 10 to 40 cm. The layer containing activated carbon and/or coke can, in preferred embodiments, also have a lower layer of activated carbon and an upper layer of coke, wherein particularly preferably the thickness of the coke layer constitutes less than 20% of the thickness of the total layer of coke and activated carbon. The coke hereby optionally used can be for example by-product broken coke. This additionally present layer of activated carbon and optionally coke surprisingly facilitates—with otherwise equal process conditions—a better reaction of the reactants chlorine and CO and therewith a reduction of the required CO excess than with normal filling with activated carbon which only extends to the end of the tubes. The CO content of the CO-containing residual gas from phosgene condensation can be reduced by this reduction of the required CO excess.

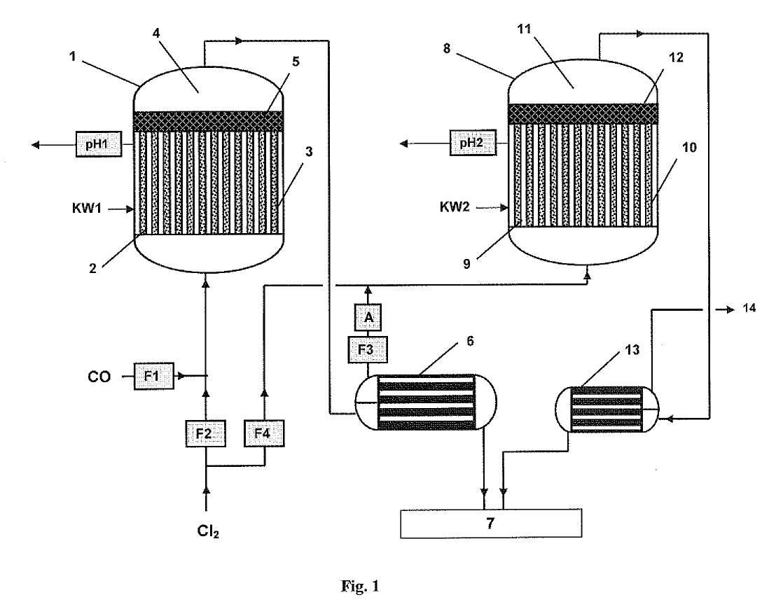

In the particularly preferred embodiment of the process according to the invention illustrated in FIG. 1., CO gas (CO) and chlorine gas (Cl₂)—in each case passed via appropriate quantity regulators F1 and F2—are thereby mixed and passed to a first phosgene generator 1 which is a tubular reactor. In this phosgene generator, cooling takes place via an indirect cooling system at cooling temperatures of less than 100° C. by conventional liquid cooling. The aqueous cooling medium KM1 flows from bottom to top through the coolant chamber 2 which flows around the reaction tubes 3. The cooling medium is subjected to a continuous pH control (pH1). The reaction tubes 3 are filled with activated carbon as catalyst. Moreover, there is in the upper gas chamber 4 of the tubular reactor a layer 5 of activated carbon with coke which has a lower layer of activated carbon with an upper layer of coke. After reaction of the CO-chlorine gas mixture in the phosgene generator, the crude phosgene obtained is passed into a first phosgene condenser 6 where phosgene is condensed out and passed to a liquid phosgene receiver 7. The gases not condensable in the first condenser 6 are passed via a quantity control F3 into the secondary generator 8. The off gas stream containing the uncondensable gases from the first condenser is thereby continuously subjected to an analysis of the CO content (A) and chlorine continuously added via a quantity control F4 in accordance with the CO content determined. The gas mixture is passed to the secondary generator 8 from below. Cooling is also carried out in the secondary generator 8 via an indirect cooling system at cooling temperatures of less than 100° C. by conventional liquid cooling. The aqueous cooling medium KM2 flows from bottom to top through the coolant chamber 9 which flows around the reaction tubes 10. The cooling medium is subjected to a continuous pH control (pH2). The reaction tubes 10 are filled with activated carbon as catalyst. Moreover, there is in the upper gas chamber 11 of the tubular reactor a layer 12 of activated carbon with coke which has a lower layer of activated carbon with an upper layer of coke. After reaction of the CO-chlorine gas mixture in the secondary generator, the crude phosgene obtained is passed into a secondary condenser 13 where phosgene is condensed out and is also passed to the liquid phosgene receiver 7. The uncondensable off gases 14 from the secondary condenser are passed to the phosgene decomposition unit.

The phosgene produced in accordance with the process according to the invention can be used to produce polymers or the precursors thereof. Typical representatives of such polymers or the precursors thereof are for example polycarbonates PC, diphenyl carbonate DPC, methylene diisocyanate MDI, toluene diisocyanate TDI and hexamethylene diisocyanate HDI.

The following examples serve to illustrate the invention by way of example and should not be regarded as restrictive.

All the references described above are incorporated by reference in its entirety for all useful purposes.

While there is shown and described certain specific structures embodying the invention, it will be manifest to those skilled in the art that various modifications and rearrangements of the parts may be made without departing from the spirit and scope of the underlying inventive concept and that the same is not limited to the particular forms herein shown and described.

### EXAMPLES

773 m³/h gaseous chlorine with a content of 99.95 vol. % chlorine, 0.01 vol. % hydrogen, 0.1 vol. % oxygen and 15 mg/m³ water and 800 m³/h gaseous carbon monoxide with a content of 97.75 vol. % carbon monoxide, 0.9 vol. % hydrogen, 0.12 vol. % oxygen, 3 mg/m³ water, 20 vol. ppm methane and 2 mg/m³ sulfur (excess pure CO based on pure chlorine of 1.2 vol. %) were mixed using a static mixer and passed through a first phosgene generator cooled with water, with reaction tubes containing activated carbon of the RB4® type from NORIT and a layer on top of this of 20 cm activated carbon with coke which has a lower layer of activated carbon with an upper layer of coke. Passing through the first generator was carried out from bottom to top. The outlet temperature at the head of the first phosgene generator was 60° C. Of the gas stream containing approx. 95 vol. % phosgene, the majority of the phosgene was condensed out in a first phosgene condenser at a temperature of −25° C. at 1.6 bar (absolute) and passed to a cooled liquid phosgene receiver.

In the remaining 38.4 m³/h gas mixture, the content of carbon monoxide was measured at 43 vol. % (16.5 m³/h). The residual content of chlorine was less than 1 ppm. In addition, inter alia 11 vol. % phosgene was contained in the gas mixture. The gas mixture was mixed with 15.7 m³/h chlorine gas, which corresponded to an approx. 5 vol. % excess of pure carbon monoxide based on pure chlorine, and passed from the bottom via a phosgene secondary generator cooled with water with reaction tubes containing an activated carbon of the RB4® type from NORIT and a layer on top of this of 20 cm activated carbon with coke which has a lower layer of activated carbon with an upper layer of coke. The outlet temperature at the head of the phosgene secondary generator was 59° C. Of the gas stream containing approx. 52 vol. % phosgene (residual content of chlorine<5 ppm), the majority of the phosgene was condensed out in a phosgene secondary condenser at a temperature of −25° C. at 1.6 bar (absolute) and passed to the same cooled liquid phosgene receiver as the phosgene from the first phosgene condenser. The residual gas stream was passed through a phosgene decomposition unit and then released phosgene-free into the atmosphere. After phosgene decomposition, the carbon monoxide content was just 0.82 m³/h, which corresponded to less than 1/20 of the quantity that was still present in the residual gas of the first phosgene condenser.

1. A process for continuously producing phosgene, comprising producing phosgene from CO and chlorine in the presence of at least one catalyst in at least one generator, condensing said phosgene in a condenser, separating off CO-containing residual gas, reacting said CO-containing residual gas with chlorine in a secondary generator to produce phosgene, condensing said phosgene produced in said secondary generator in a secondary condenser, and separating off uncondensed residual gas.

2. The process of claim 1, wherein addition of chlorine into said secondary generator is controlled by analysis of the CO content of the CO-containing residual gas.

3. The process of claim i, wherein addition of chlorine into said secondary generator is controlled such that the gas mixture added to said secondary generator has a CO excess of at least 1.5 volume % based on added chlorine.

4. The process of claim 1, wherein said uncondensed residual gas is discharged from the process and passed to a phosgene decomposition unit.

5. The process of claim 1, wherein said catalyst is activated carbon and/or coke.

6. The process of claim 1, wherein said generators and/or secondary generators are heat exchangers in the form of tubular reactors.

7. The process of claim 6, wherein said tubular reactors have parallel vertically arranged reaction tubes wherein a coolant circulates in the coolant chamber laterally surrounding the reaction tubes and which terminate at their upper and lower end in each case in an upper and lower gas chamber of the tubular reactor, wherein the reaction tubes of the tubular reactor are filled with activated carbon and the upper gas chamber of the tubular reactor additionally contains on the reaction tubes a layer of activated carbon and/or coke.

8. The process of claim 7, wherein said reaction tubes are filled at the lower end with catalytically non-active materials and on them have a layer of activated carbon.

9. The process of claim 1, wherein several generators are used in parallel operation.

10. The process of claim 1, wherein said phosgene condensed in said condenser and in said secondary condenser is collected in a common liquid phosgene receiver.
